(19) United States
(12) Patent Application Publication
(10) Pub. No.: US 2006/0058700 A1
(43) Pub. Date: Mar. 16, 2006

(54) Patient sedation monitor

(76) Inventors: Dominic P. Marro, North Andover, MA (US); Henry R. Ortega, Hampstead, NH (US)

Correspondence Address: BRIAN R. WOODWORTH, 275 N. FIELD DRIVE, DEPT. NLEG BLDG H-1, LAKE FOREST, IL, 60045-2579, US

(21) Appl. No.: 11/211,349

(22) Filed: Aug. 25, 2005

Related U.S. Application Data
(60) Provisional application No. 60/604,799, filed on Aug. 26, 2004.

(51) Int. Cl. A61B 5/05

(52) U.S. Cl. 600/554

(57) ABSTRACT

A system for determining a patient's level of sedation. The system includes a glabellar stimulator constructed to generate an electrical stimulus. An electrode is electrically connected to the glabellar stimulator, the electrode being constructed to deliver the electrical stimulus from the glabellar stimulator to a patient. The system further includes a patient module constructed to detect an eyeblink response of a patient following delivery of the electrical stimulus to the patient. The patient module is constructed to generate a signal indicative of at least one parameter of the eyeblink response, wherein the at least one parameter of the eyeblink response is indicative of a patient's level of sedation.

### CROSS REFERENCE TO RELATED APPLICATION

This application claims priority from U.S. Provisional Patent Application No. 60/604,799, filed Aug. 26, 2004.

### FIELD OF THE INVENTION

The current invention relates to a system for the differentiation between hypnotic and paralytic states of a patient undergoing medical anesthesia or sedation. It further relates to the use of electrophysiological signals to identify and differentiate such states. More particularly, it relates to use of physiological signals to distinguish between natural sleep, on the one hand, and anesthesia and sedation on the other. The invention further relates to the use of electroencephalographic signals, in concert with other physiological and electrophysiological signals, to identify and differentiate such states.

### BACKGROUND OF THE INVENTION

In current medical practice, patients are placed under general anesthesia during invasive surgery. In post-surgical and other medical situations, particularly in an intensive care unit (ICU), patients are sedated although not fully anesthetized. Commonly administered anesthetic and sedative drugs cause a patient to lose consciousness and/or sensation, or at least to have diminished consciousness and/or sensation. An anesthesia practitioner monitors the patient's state of awareness by means of clinical signs known empirically to provide useful and reliable information about the patient's state of awareness or unconsciousness.

Post surgery, and in other medically required circumstances, a patient is admitted to an ICU for close monitoring of condition and for relevant treatment. While in the ICU a patient is often sedated, sometimes heavily, sometimes lightly. It is important to maintain the ICU patient at an appropriate level of sedation. Drugs commonly used to manage patient sedation include hypnotics, anxiolytics, and analgesics. One drug used to manage patient sedation is PRECEDEX dexmedetomidine.

In all of the above situations, frequent assessment of the patient's state of anesthesia or sedation is crucial. The need for patient sedation monitoring also exists in office based surgery, ambulatory surgery, and recovery rooms.

With respect to induced full or partial hypnotic states, clinicians typically monitor the patient's state visually using one of several known scales that are based on patient characteristics. Sedation monitoring currently is accomplished by using one or more of ten subjective scoring systems. These scoring systems include, but are not limited to, Ramsey Sedation Scale, Riker Sedation Agitation Scale, Richmond Sedation Scale, Motor Activity Assessment Scale, Bion Scale, Glasgow Coma Scale, and others. When properly used, these scoring systems have proven to be an effective way to decrease mortality and morbidity in the ICU, and, particularly with ventilated patients, decrease the amount of sedative drugs used, shorten the stay in the ICU, decrease incidence of ICU psychosis, and improve patient comfort.

These scoring systems have a number of drawbacks in common, including:
1. Intervention on the part of a clinician is required in order to complete the assessment.
2. Measurement of the patient's response to certain stimuli is required.
3. The stimulus provided by the clinician is subjective in nature.
4. The clinician's observation of the response is subjective in nature.
5. Record keeping is manual.

Due to the inherent subjectivity of these tests, it is difficult to provide a predictable, accurate measurement of the patient's depth of sedation. This limitation underscores the need for an automatic sedation monitor that provides an objective measurement regardless of the clinician administering the test. Because clinicians are accustomed to measuring depth of sedation using the known, subjective tests, it is advantageous that the automatic sedation monitor, at least in one embodiment, be scaled to one of the more common and familiar sedation scales.

The most widely used anesthesia/sedation scale is the Ramsay Sedation Scale (RSS). This scale is simple and relatively straightforward for the clinician to apply, although imprecise and subjective for the reasons discussed above. The stages and indications of the RSS are shown in Table 1:

TABLE 1

| Score | Description | Definition |
|---|---|---|
| 1 | Awake | Patient anxious and agitated or restless or both |
| 2 | Awake | Patient cooperative, oriented, and tranquil |
| 3 | Awake | Patient responds to commands only |
| 4 | Asleep | A brisk response to external stimulus |
| 5 | Asleep | A sluggish response to external stimulus |
| 6 | Asleep | No response to external stimulus |

As the table indicates, the Ramsay Scale is divided roughly into “awake” states, stages 1 through 3, and “asleep” states, stages 4 through 6. “Asleep” in this context means either (i) normal sleep; or (ii) anesthetized or heavily sedated, i.e., a chemically induced “sleep.” One of the problems addressed by anesthesia/sedation monitor of the present invention is that of distinguishing between normal sleep and chemically induced sleep. The Ramsay Scale defines sleep at an RSS of 4, with a brisk response to external stimulus. The most common external stimulus used for this purpose is a glabellar tap, which provokes an eyeblink response (see below).

As noted, it is desirable to have an objective measurement of the level of anesthesia or sedation of a patient, possibly based on the RSS scale or another known sedation scale, so as not to have to rely on the subjective impressions of clinicians. Systems for measuring depth of anesthesia/sedation have been developed using EEG signals, generally in combination with other signals, to monitor anesthesia, sleep, and other states on the consciousness-unconsciousness continuum. Representative examples include, but are not limited to, Kaplan et al., U.S. Pat. No. 5,813,993, issued Sep. 29, 1998; Maynard, U.S. Pat. No. 5,816,247, issued Oct. 6, 1998; Kangas et al., U.S. Pat. No. 5,775,330, issued Jul. 7, 1998; John, U.S. Pat. No. 5,699,808, issued Dec. 23, 1997; John, U.S. Pat. No. 4,557,270, issued Dec. 10, 1985; John, U.S. Pat. No. 4,545,388, issued Oct. 8, 1985; Prichep, U.S. Pat. No. 5,083,571, issued Jan. 28, 1992; and John, U.S. Pat. No. 6,067,467 issued May 23, 2000.

Commercial ventures have developed practical systems for monitoring patient anesthesia/sedation state. Representative examples include a patient state analyzer (SEDLine) manufactured by Physiometrix, Inc., the analytical aspect of which is described in Ennen, et al., U.S. Pat. No. 6,317,627, issued Nov. 13, 2001, and incorporated herein by reference in its entirety, and a system manufactured by Aspect Medical Systems, Inc. The Physiometrix SEDLine analyzer is a sedation monitor that uses spectral and temporal measurements processed from the patient's EEG to estimate a level of hypnosis or sedation. It produces a measure called the patient state index (PSI). The Aspect Medical system incorporates technology described in a series of patents of which Chamoun, U.S. Pat. No. 5,010,891, issued Apr. 30, 1991, and Chamoun, et al., U.S. Pat. No. 5,458,117, issued Oct. 17, 1995, are representative examples. The methods therein described make substantial use of a calculation of bispectral (BIS) indices of consciousness and anesthesia.

The previously described scoring systems can be used in conjunction with an EEG-based anesthesia and sedation monitor to provide an objective measurement of sedation level estimate and to show trends in the patient's level of anesthesia and sedation.

Although commercially available monitors are frequently trained against the Observer's Assessment of Alertness and Sedation scale (OAAS), they cannot readily differentiate between natural sleep induced hypnosis and chemically induced hypnosis. Although a computed hypnotic state parameter may be accurate, a patient who is merely asleep will respond rapidly to a provocative stimulus, whereas a patient with the same computed level of drug induced hypnosis will not. (If this were not true, people would not wake up to their alarm clock and there would be many more wake-ups during surgical procedures.) For example, an index of 40 for the SEDLine analyzer and 50 for the BIS monitors would represent ideal sedation under most circumstances for drug induced sedation. However, these numbers are also commonly obtained from patients enjoying normal sleep.

For a patient that is merely asleep and not chemically sedated or only lightly sedated, the patient state index or the BIS index would likely rise after an external stimulus is applied, but the value of these indices as a predictor of a response assumes prior knowledge of the sedative drugs, if any, being administered to the patient. A desired characteristic of a sedation monitor would be to eliminate the need for such a-priori drug information. However, currently no automated system for scoring patients against a validated sedation scoring system exists that provides a clinician the ability to differentiate between arousable sleep and non-arousable, drug-induced hypnosis.

One of the most common external stimuli used to assess whether a patient is merely asleep or is chemically sedated or anesthetized is the glabellar tap. The glabellar tap is a primitive reflex reaction in which the eyes blink if an individual is tapped lightly directly between the eyebrows. This reflex is observed whether the eyes are open or closed. An automated indicator of response to a glabellar tap, or even better to a simulated glabellar tap, is highly desirable.

### SUMMARY OF THE INVENTION

The glabellar tap monitoring system of the present invention involves the application of a specific provocative electrical stimulus to the patient and an electronic observation of the presence or absence of a blink reflex. Automation of this assessment requires the presentation of an electrical stimulus through an auxiliary circuit, usually referred to herein as the “glabellar stimulator,” and the monitoring of the patient's response to the stimulus, particularly the patient's eyeblink amplitude and the patient's eyeblink response latency. The stimulus is delivered as an objective, repeatable stimulus delivered electronically either automatically or upon the demand of the clinician, e.g., through the use of a push-button activator.

The fully automated version of this invention includes equipment necessary for the electronic measurement of the patient's eyeblink amplitude, eyeblink latency, and morphology (e.g., the system described in U.S. Pat. No. 6,317,627), equipment necessary for calculating a response value based upon these electronically measured parameters, and a display for communicating the response value to a clinician. The glabellar stimulator can be integrated with a known EEG monitoring systems, such as that described in U.S. Pat. No. 6,317,627, or can be a stand-alone system designed to operate functionally in combination with such an EEG monitoring system. In the EEG system disclosed in U.S. Pat. No. 6,317,627, a plurality of electrodes are mounted on the patient's forehead, with at least one electrode, preferably the ground electrode, located just above an anatomical point called “the Nasion,” The Nasion is the valley or recessed area (as seen in profile) that is just below the eyebrows, generally considered to be where the nose “starts”. In most patients the Nasion is at the same level as the tips of the upper eyelashes. The Nasion is a reference point that can be used to locate electrodes associated with an EEG monitoring system.

The electronic glabellar tap stimulating and measuring system of this invention automates the delivery of a precise electrical stimulus that is independent of patient contact impedance. The system accomplishes this task by delivering a predetermined amount of charge from the stimulus circuit. The stimulus magnitude is independent of contact impedance by virtue of an arrangement in which a charge control comparator increases the pulse duration for a given preset stimulus magnitude and a higher contact impedance, resulting in the desired total charge being transferred to the patient. The system provides a continuous pulse train of mono-phasic or multi-phasic pulses. The system may also be programmed to deliver a train-of-four or a double burst stimulation pattern for assessment of drug-induced paralysis.

The embodiment of the present invention disclosed herein is calibrated to the Ramsay Sedation Scale (RSS) because of the RSS system is very familiar to many practitioners. However, it is to be appreciated that the present invention can be calibrated to any of the known sedation scales, and that the present invention can be parameterized to a new sedation scale, either one specifically designed for use with the system of the present invention or one that has applicability beyond the present invention.

The stimulus circuitry used in connection with the present invention can be actively charge-balanced to produce an approximately zero net charge transfer, that is, a substantially charge neutral electrical stimulus pulse or pulse train, within the glabellar stimulator blanking period. This feature contributes to achieving a near zero offset at the amplifier input, thereby contributing to maximum attenuation of the stimulus pulse artifact. Zero net charge, however, does not mean zero net energy. The stimulus current, independent of its sign, provides “stimulus energy”, which means energy as sensed by the patient's peripheral nervous system, not the calculated net physical energy delivered by the pulse generator. The patient's response, although non-linear, is a monotonically increasing function of the “stimulus energy”. For the most part, the difference between the energy delivered by the stimulus pulse generator and the stimulus energy is accounted for by the I²R losses from the electrodes.

The circuitry of the current invention is designed to be integrated with an EEG amplifier where, within milliseconds of the stimulus, EEG and eyeblink signals are processed. The EEG and eyeblink signal acquisition can be temporarily disabled while the stimulus is being applied to avoid unwanted transient artifacts caused by the stimulus pulse. The circuitry is also capable of being programmed to create train-of-four and tetanus pulses for paralysis monitoring.

### BRIEF DESCRIPTION OF THE DRAWINGS

FIG. 1 is a functional diagram of an EEG data acquisition system and display with glabellar stimulator capability;

FIG. 2 depicts a shunt configuration at a patient module preamplifier stage;

FIG. 3 depicts a functional circuit diagram for the stimulation pulse generation circuitry of the present invention;

FIG. 4 depicts glabellar stimulus pulse morphologies and characteristics flowing from those pulse shapes; and

FIG. 5 depicts a schematic of the stimulation pulse of the pseudo-glabellar tap and the variation of the response eyeblink amplitude and latency with increasing sedation.

### DETAILED DESCRIPTION OF THE PREFERRED EMBODIMENT

As noted above, the glabellar tap is a reflex wherein a person's eyes blink if the individual is tapped lightly between the eyebrows. It has been determined that an electrical stimulus of the correct amplitude and duration, and of the correct pulse shape, will provoke a pseudo-glabellar tap blink reflex that varies in a predictable way and that produces response parameters, i.e., presence and magnitude of eyeblink, that can be detected and measured to generate an objective determination of the patient's depth of sedation. The presence and magnitude of the eyeblink response can be measured using an analytical system of the type disclosed in U.S. Pat. No. 6,317,627, optionally with modifications to the software for improved performance. Other EEG based monitoring systems for detecting and measuring the presence and magnitude of eyeblink can be configured used in conjunction with the present invention.

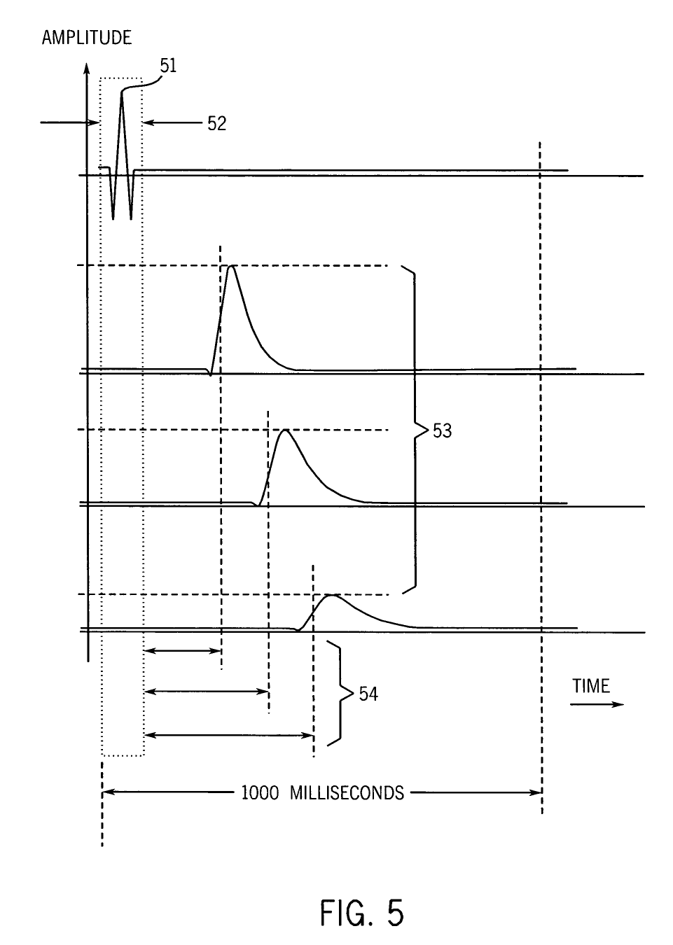

In order for an eyeblink event to be identified and scored for the waveforms depicted in FIG. 5, certain conditions must be met. The eyeblink event is detected, for example, in the manner described in Ennen, et al., U.S. Pat. No. 6,317,627, column 9, line 26 to column 10, line 8. The detection of an eyeblink sets a detection window beginning at the end of the stimulus-blanking period and ending when the eyeblink is detected, but not later than 1000 milliseconds after the stimulus. Within the detection window, the peak amplitude is determined by the difference between the baseline signal level captured just prior to the blanking period and the maximum amplitude of the signal as depicted at 53 in FIG. 5. The peak amplitude detector, using methods well known in the art of digital signal processing, acquires both the peak amplitude and the sample count. The eyeblink latency is the difference between the sample count associated with the peak amplitude and the stimulus. The eyeblink amplitude in microvolts (peak) must be greater than a predetermined or adaptive threshold. The eyeblink latency (milliseconds) must be within the detection window and must be less than a predetermined or adaptive threshold.

Each or a selected combination of the derived parameters of eyeblink amplitude and latency will produce an output, which when compared to predetermined or adaptive thresholds, are used to estimate the Ramsey Sedation Score. The Ramsey Sedation Score or any equivalent processed value can be displayed as a dimensionless metric such as used in RSS or RASS, or probability score representative of the probability that the person is responsive or non-responsive.

In alternative embodiments of the present invention, alternative physical and electrophysiological methods for detecting eyeblinks are utilized. One alternative utilizes a properly placed photoreflective sensor to detect eyeblinks. Although the photoreflective sensor is electrically isolated from stimulus pulse artifact and can detect both the presence of an eyeblink and the eyeblink latency, the accuracy of the photoreflective sensor's amplitude measurements may vary dependent on sensor placement. Other optical systems such as those used in headgear designed to detect drowsiness for certain task monitoring applications also can be used in conjunction with the present invention to detect eyeblinks.

The system of the instant invention replaces the stimulation of a mechanically applied glabellar tap with an electrical stimulus pulse. This pseudo-glabellar tap system uses, in one embodiment, a standard frontal (forehead) array of electrodes, e.g., conducting gel electrodes, to transmit electric pulses to appropriate locations on a patient's forehead. Preferably these locations are selected from known locations on the patient's forehead, e.g., the F8, Fp1, Fp2, F7, Afz, and Fpz locations, which are used to collect EEG input in the Physiometrix SEDLine system. However, it will be appreciated that other designations or locations can be used with various EEG monitoring systems.

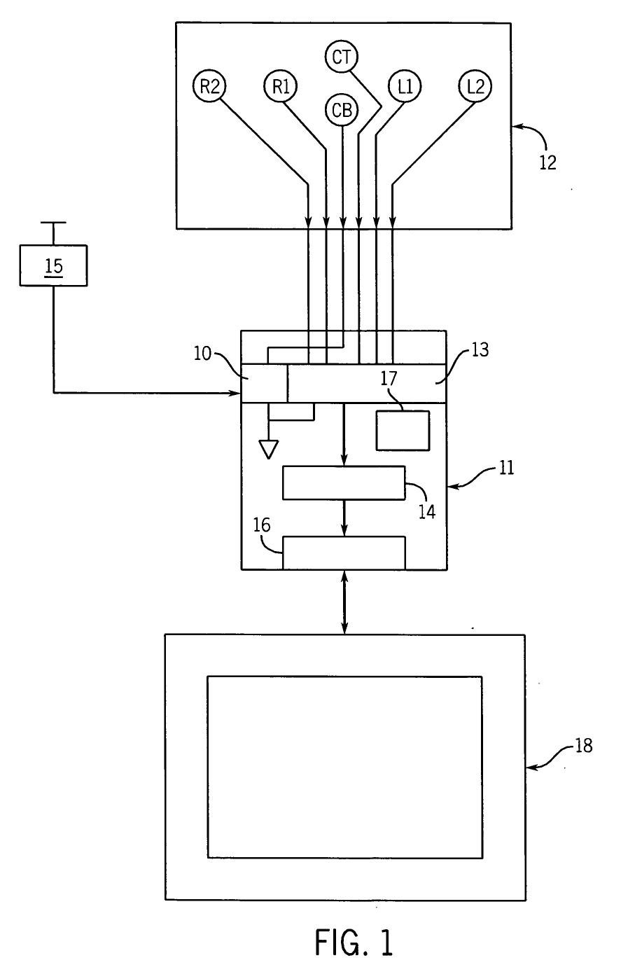

FIG. 1 shows the overall architecture of the system including the glabellar stimulator generator. Glabellar stimulator 10 can be contained integrally in patient module 11, or it can be separate from patient module 11. Patient module 11 is constructed as described in U.S. Pat. No. 6,430,437, which is incorporated by reference herein in its entirety. Electrode array 12 positioned on the patient's forehead sends signals to preamplifier-multiplexer 13 of patient module 11. The patient module 11 includes an analog-to-digital converter 14 and an isolated serial input output segment 16. Glabellar stimulator 10 may include a push button module 15 which enables a clinician to initiate pulse trains manually rather than allowing glabellar stimulator 10 to initiate pulse trains automatically.

Serial input output segment 16 sends converted signals to host instrument 18. As previously indicated, host instrument 18 can be configured per the system described in U.S. Pat. No. 6,317,627.

The stimulus pulses generated by the glabellar stimulator circuit of the present invention can approach 100 volts, and thus can be more than six orders of magnitude larger than the physiological responses being measured. For this reason, the system of the present invention preferably includes a system to attenuate this voltage by blocking the amplifiers' input during delivery of the stimulus (blanking period), by attenuating all signal inputs, and by minimizing the residual charge or charge transfer left on the second stage filter.

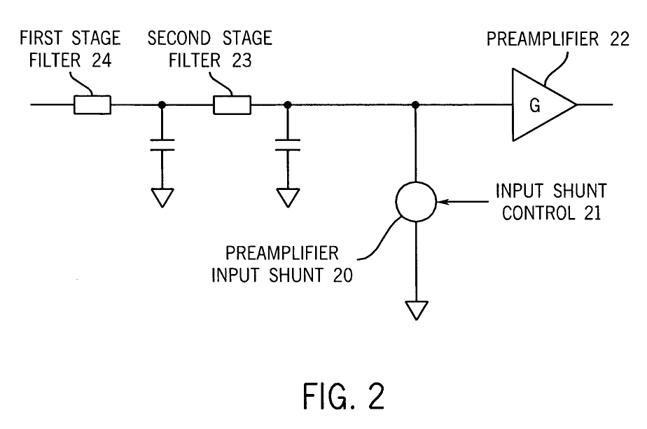

As illustrated in FIG. 2, because transients produced by the stimulus could swamp EEG, eyeblink, and/or EMG signals for several seconds, and thereby interfering with proper eyeblink detection, glabellar stimulator 10 contains a subcircuit that automatically disables the patient module preamplifier just before, during, and just after the transmission of the stimulus pulse by shunting the preamplifier input to ground during the glabellar stimulator blanking period. With reference to FIG. 2, preamplifier input shunt 20 is controlled by the input shunt control 21 provided by pulse sequence logic 33, which, in turn, is triggered by the initiation of a pulse train in glabellar stimulator 10. Preamplifier input shunt 20 causes any signal coming from second stage filter 23 to be shorted to ground. This action diverts most of stimulus pulse energy away from the patient module preamplifier and subsequent filter stages and thereby minimizes analyzer input signal corruption. Other circuit elements activate the shunt as indicated in a separate column in Table II below.

During the shunt period the generation of the patient state index using previously transmitted signals can continue uninterrupted while the stimulus pulse is transmitted. The patient's response is analyzed after the shunt is reopened and incoming signals reach the preamplifier again. Blanking the amplifier in this manner makes it possible to detect eyeblinks within milliseconds of the stimulus.

The shunt by itself, however, may only attenuate the pulse voltage that reaches the preamplifier by a factor of approximately 100 to 1. For this reason, it may be necessary to provide additional protection from the pulse stimulus. Protective circuitry provided in patient module 18 can provide an additional 50 to 1 attenuation factor. As explained more fully below, approximately zero net charge transfer in the glabellar stimulator pulse train provides an additional attention factor of approximately 10-20 to 1, and common mode rejection of the residual pulse artifact that persists as an offset voltage can achieve an additional attenuation factor of approximately 10-20 to 1.

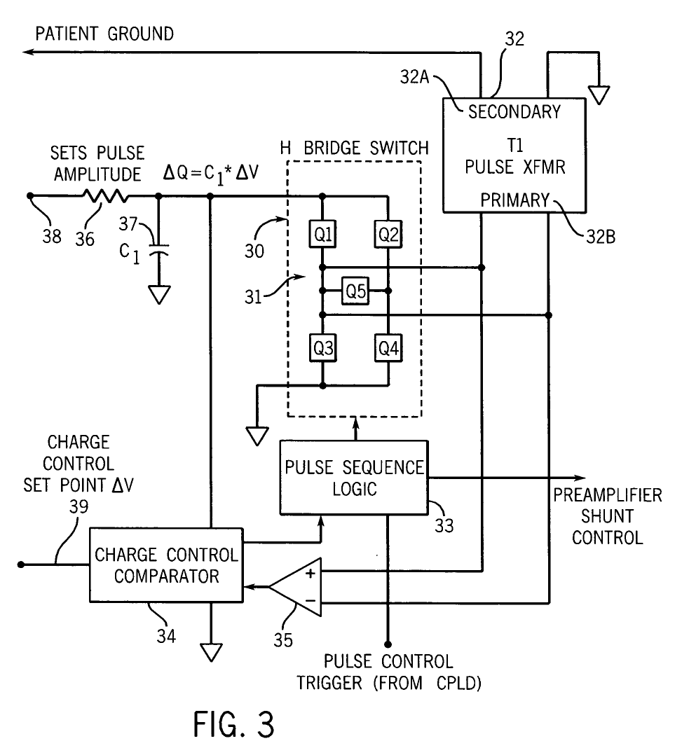

Referring to the circuit diagram of FIG. 3, the secondary 32A of transformer T1 is connected in series with a patient return lead and an amplifier signal return. The transformer primary 32B is connected to H-bridge switch 30 and to H-bridge shunt 31. H-bridge shunt 31 can include a plurality of switches. In the embodiment of the present invention depicted in FIG. 3, H-bridge shunt 31 includes five switches, Q1, Q2, Q3, Q4, and Q5. These circuit elements can be basic solid state switching elements, for example field effect transistors, MOSFETS, bipolar transistors, or other solid state switching elements.

During routine patient monitoring using the circuitry depicted in FIG. 3, all four branches of the H-bridge are open and the H-bridge shunt is closed. The H-bridge shunt is configured to provide a low impedance ground connection between the patient and the amplifier through the transformer secondary 32A by shorting the primary 32B during normal EEG monitoring. When desired, either by a button push or by automatic scheduling, a stimulation pulse is generated, while simultaneously (a) the H-bridge shunt is opened; and (b) diagonally opposing branches of the H-Bridge are closed generating a voltage impulse on the primary and secondary of T1.

The preamplifier blind period is not created by this H-bridge shunt but rather is created by the preamplifier input shunt 20 described above and illustrated in FIG. 2. The input shunt is closed while the H-bridge shunt is open.

Pulse polarity is determined by the set of opposing H-Bridge branches that are closed. In the embodiment of the present invention depicted in FIG. 3, a Q1 and Q4 combination produces a pulse of positive polarity while a Q2 and Q3 combination produces a pulse of negative polarity. Capacitor C1 having a capacitance CX is charged to voltage V1 through resistor R1 to achieve a charge of Q_{c} (where Q_{c}=Cₓ×V1 coulombs). The RC time constant is preferably set such that tRC is short enough to recharge to a level of >99% of V1 in less than 500 milliseconds after maximum controlled discharge.

The requisite pulse sequence logic is pre-programmed for a plurality of selectable pulse sequences. The stimulus pulse mode can be selected from a menu associated with host instrument 18. Host instrument 18 sends a command to a programmable logic array (PLA) 17 in the patient module, thereby setting its internal logic to initiate (upon command) the desired pulse sequence. The pulse-timing parameters are stored in the PLA 17. The stimulus pulse command can be initiated by depressing an external pushbutton 15, or by a timer in host instrument 18 that has been set by the user to check patient status at predetermined intervals.

The system of the present invention preferably is configured to monitor total charge in order to deliver the desired (relative, not absolute) stimulus energy. The net stimulus effect is independent of the sign and proportional to stimulus energy (in turn proportional to I²). In other words, the stimulus effect does not net out to zero while the system is driving the net stimulus pulse charge to zero.

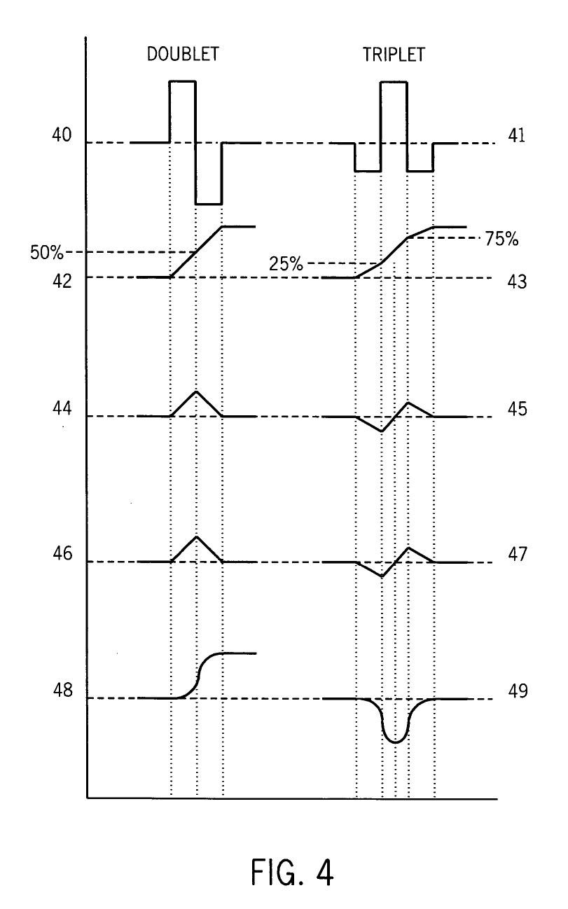

The charge control comparator 34 depicted in FIG. 3 includes three comparators, i.e., comparators 1-3. Comparator 1 monitors voltage changes on C1 and is used to control the total energy delivered to the patient by the stimulus pulse. For a biphasic pulse, comparator 1 triggers the pulse sequence logic 33 to invert the phase of a biphasic stimulus pulse when 50% of the programmed stimulus energy has been delivered. (See 42 in FIG. 4.) The voltage change on C1 is proportional to the product of the current and time divided by its capacitance. Total stimulus energy is controlled by setting a charge control set point for comparator 1 to a voltage below V1 that is reached when 50% of the intended stimulus energy has been delivered to the patient. The phase reversed stimulus pulse then terminates when the output of the net charge integrator 35 returns to the reference value just prior to the stimulus pulse as shown at 44 in FIG. 4. This terminates the stimulus pulse at zero net charge and 100% of the intended stimulus energy. Stimulus pulse phase reversal and termination are accomplished by control signals from the charge control comparator to the pulse sequence logic 33 in FIG. 3. Selected pairs of switches as described in Table II open and close in response to these commands to generate a biphasic pulse.

A triphasic pulse sequence with zero net charge can be produced in a similar fashion. Comparator 1 will trigger the pulse sequence logic 33 to invert the phase of a triphasic stimulus pulse when 25% of the programmed stimulus energy has been delivered. Comparator 3 will trigger the pulse sequence logic 33 to invert the phase of a triphasic stimulus pulse when 75% of the programmed stimulus energy has been delivered. This final phase reversed stimulus pulse then terminates when the output of the net charge integrator 35 returns to the reference value just prior to the stimulus pulse as shown at 45 in FIG. 4.

For a given preset stimulus magnitude and higher contact impedance, the charge control comparator increases the pulse durations resulting in the same total charge being transferred to the patient. A voltage V1 at resistor 36 is set to ensure that the primary pulse magnitude at the transformer primary 32B times the turns ratio can produce a voltage of approximately 60 volts at the transformer secondary 32A. The transformer also provides for patient safety by providing isolation between active electronic circuitry and patient applied parts.

In an idealized case, the charge delivery efficiency of the stimulator is 100%. The very short switching times and low RON for the H-Bridge and the low primary resistance for T1 with optimized ET constant ensure optimum efficiency. The voltage drop on C1 is a reflection of the total charge transferred.

Table II identifies and describes applicable circuit states:

TABLE II

| State | Q1 & Q4 | Q2 & Q3 | Q5 | Input Shunt |
|---|---|---|---|---|
| Data Acquisition | Open | Open | Closed | Open |
| +Pulse | Closed | Open | Open | Closed |
| −Pulse | Open | Closed | Open | Closed |

Basic stimulus pulse performance requirements related to circuit design of the pulse generator are addressed as follows:

| | | |
|---|---|---|
| 1. | Pulse magnitude: | 0 to +/−40 milliamperes |
| 2. | Pulse duration: | 100 to 800 microseconds |
| 3. | Pulse morphology: | Biphasic & Triphasic. |

The pulse generation circuit can be constructed such that it is capable of generating a plurality of pulse types beyond the glabellar stimulation pulses of the current invention. For example, the pulse generation circuit can be constructed to transmit a series of provocative stimuli separated by variable intervals of short duration. Specific appropriate pulse shapes and durations can be preprogrammed into the system as shown in FIG. 4. Pulse sequence logic can be pre-programmed to have the required switch timing and states to produce the requisite patterns for glabellar stimulator pulses as well as for Train-Of-Four, Double Burst, Tetanus, and other desired pulse patterns. Higher stimulation currents can also be provided for a supra-maximal stimulus, which is in normal practice the basis of Train-Of-Four measurements. The time constant constraint referred to above ensures that consecutive pulses during a Train-Of-Four sequence will be of the same amplitude.

The system of the current invention is capable of generating the bi- and tri-phasic stimulation pulses shown in FIG. 4. The pulse shape is configured to minimize unwanted impact on response measurement systems. The pulse shape is preferably configured to minimize residual offset in preamplifier filters. (As noted above, the preamplifier shunt circuit element of FIG. 2, by blinding the preamplifier during pulse generation, provides partial insulation from the potentially overpowering effect of the stimulation pulses. However, as noted, additional reduction in the effect of pulse transmission may be necessary.)

The Physiometrix SEDLine preamplifier has a high level of immunity to environmental, physiological, and procedural interference, in part by virtue of filtering. (A description of the preamplifier and related circuitry appears in U.S. Pat. No. 6,430,437.) The Physiometrix SEDLine filtering configuration is a multistage filter comprising part of the SEDLine's anti-aliasing system.

As with the Physiometrix SEDLine system, the input stage for physiological monitoring systems in general has single or multi-stage filters. However, different designs of the input stage may require correspondingly different pulse morphology to achieve comparable results in a different filter configuration.

When small electrophysiological signals such as EEG, EMG and EOG are being monitored concurrently using the same leads as the stimulus, the effective net charge transfer should be as close to zero as possible in order to minimize contamination of the incoming signals by the stimulus pulse. A stimulus pulse several orders of magnitude larger than the physiological signals being monitored gives rise to a significant residual offset in a preamplifier stage proportional to the net charge divided by the filter capacitance of that stage. As spelled out more fully below, the use of the pulse morphologies shown in FIG. 4 with zero net charge transfer minimizes these residual offset voltages, permitting resumed detection of eyeblink responses or other low level signals within milliseconds of the stimulus pulse.

With reference to FIG. 4, potentially usable pulse morphologies include the doublet pulse shape 40 and the triplet 41. The total charge parameter for each is shown in 42 and 43. The net charge parameter is 44 and 45. Both pulse shapes have appropriate net charge transfer. However, it is only at the second filtering stage of preamplifier 23 that the adverse effect of the doublet morphology is shown (see 48). At the second filter stage 23 (shown in FIG. 2), the net residual offset is zero (see 49). When the Physiometrix SEDLine system is constructed in accordance with the present invention, the preferred pulse shape is the triphasic pulse. However, in other embodiments of the system of the present invention having different filtering configurations (when compared to the Physiometrix SEDLine system), either the doublet shape or other shapes may be appropriate.

In addition to the basic shape configuration, two overall parameters of the charge transfer pulse amplitude and shape are important in the downstream functioning of the pseudo-glabellar tap electrical stimulation system. The first is the stimulus pulse net charge. The net charge parameter is the integral over time of the (signed) value of the current flow, positive and negative, that the system delivers. In order to minimize effects on downstream electronics, the pulse parameters are manipulated so that the Net Charge is as close to zero as is practicable. Zeroing out the net charge produces the electrical equivalent of a glabellar tap while minimizing the residual stimulus pulse artifact due to the net charge at the preamplifier.

The second important parameter is the stimulus pulse total charge. The total charge represents the integral of the absolute value of the stimulus current. Use of the word “Total” refers to the integrated value of the current of either sign, that is, the total charge in and out, that flows through the patient. The voltage change V1 ΔA) measured at 38 in FIG. 3, the capacitance C1, and the pulse duration determines stimulus pulse total charge. The voltage V1, the capacitance C1, pulse phase and duration determine stimulus pulse net charge. Total charge and net charge integrator initial conditions are set to zero during normal data acquisition, and are enabled during stimulus pulse generation.

The physiological stimulus level is a function of the pulse amplitude and duration. It is not entirely a function of, or proportional to, the stimulus pulse total energy, which is proportional to the time integral of the square of the current, but rather is a function of the integral of the absolute value of the current over time. It has been found that the magnitude of the pseudo-glabellar tap response is a monotonically increasing function of the total charge parameter, as defined above.

The stimulus circuitry is connected in series with the patient signal ground lead, preferably located just above the Nasion. In the case of use of the Pysiometrix SEDLine system and the Physiometrix frontal array, the ground lead located just above the Nasion delivers the full stimulus current, while the remaining applied electrodes (5) each return approximately 20% of the delivered stimulus current. This configuration ensures proper focus of the stimulus for the pseudo-glabellar tap just above the Nasion.

Pulse current and duration can be controlled separately. (See FIG. 3.) The pulse current is proportional to the pulse amplitude, which is controlled. The patient contact impedance is not controlled, but is compensated for by controlling the total charge transferred. Since the stimulus magnitude is proportional to the total charge transferred, the stimulus magnitude can be controlled over a wide range of contact impedances by setting the pulse amplitude and measuring the charge transferred to the patient by comparing the voltage drop on C1 to a charge control set point. When the voltage drop on C1 equals a predetermined set point magnitude, the desired stimulus magnitude has been achieved. Within the range of pulse duration needed for proper stimulation, the stimulus magnitude is proportional to the total pulse coulombs. With this invention, it is only necessary to set the magnitude of the desired charge (in coulombs). When the targeted amount of coulombs is transferred to the patient, the pulse is terminated.

Eyeblinks arising from the glabellar reflex will occur within a window of 50 to 1000 milliseconds after the stimulus pulse. The properties of the stimulus pulse (as described above) and the EEG, EOG, and EMG signal acquisition process (as described below) produces a reliable measurement of eyeblink response.

As shown in FIG. 5, the eyeblink reflex has a predictable morphology and latency. The morphology and latency of the eyeblink reflex changes in a predictable way with increased levels of sedation. A schematic version of this variation is shown. As previously noted, the Physiometrix SEDLine preamplifier (and therefore the input) are in a blanking period 52 during the period of the stimulus pulse 51. As shown in FIG. 5, eyeblink amplitude 53 decreases with increasing sedation, and eventually, at even higher sedation levels, no eyeblinks are detected. In addition, eyeblink latency 54 increases with increasing sedation. The eyeblink measurement system and technique of the currently preferred embodiment estimates eyeblink parameters including both amplitude and latency.

These measurements are compared to preset thresholds arrived at empirically. The eyeblink response to the pseudo-glabellar stimulator is measured and scored to determine equivalent RSS value in the range of levels 3 through 6. Combining the derived equivalent RSS value with, in the PSI from the patient state analyzer helps to differentiate between natural sleep and drug sedation.

The system of the present invention can be constructed utilizing an EEG-based eyeblink detector embodiment substantially similar to that described in U.S. Pat. No. 6,317,627, incorporated herein by reference. The function of eyeblink detection according to Ennen, et al. is described at Col. 9, line 26, through Col. 10, line 7. Eyeblink measurement parameters include amplitude and latency. These measurements are compared to preset thresholds that are arrived at empirically.

The system of Ennen, et al. can be modified to provide improved eyeblink discrimination. The eyeblink signal can be optimized in the presence of background EEG, EMG, and noise by summing the contra-lateral bipolar electrode pairs, for example, (Fp2-F8)+(Fp1-F7). Changes in the eyeblink signal profile that are a function of where the eyes are pointing are minimized by summing the contra-lateral bipolar electrode pairs, for example: (Fp2-F8)+(Fp1-F7).

The bipolar measurements (Fp2-F8)+(Fp1-F7) provide additional reduction in residual common mode energy when the stimulus is presented between the ground and reference leads. The signal represented by (Fp2-F8)+(Fp1-F7) is analyzed as described above. The eyeblink detector as described in U.S. Pat. No. 6,317,627 utilizes the sum of the contra-lateral bipolar electrode pairs referred to above.

Because most clinicians are skilled in and comfortable with the Ramsay Sedation Scale and its application, the embodiment of the present invention discussed herein converts the eyeblink parameter output to an RSS number. This scale is calibrated by clinical benchmarking. The method of measurement of eyeblink amplitude and latency are discussed above. These two parameters establish a bivariate function of sedation level. Using any of a number of techniques, including but not limited to the discriminant function technique referenced in U.S. Pat. No. 6,317,627, these values can be combined into a single discriminant score that is a monotonic function of sedation level. This in turn can be scaled, preferably by a monotonic function, to a Ramsay Sedation Scale value. Alternatively either the single parameter or the raw amplitude and latency values are tabulated, compiled in a database of eyeblink and latency measurements, and then compared statistically with clinician estimates of RSS. The resulting collection of clinical data in comparison to measured parameters enables the establishment of a functional relationship between either the raw parameters or a discriminant score and the RSS. By comparing the means, or the weighted combination of the means, of amplitude and latency measurements at clinically estimated RSS scores, an RSS equivalent scale is provided.

The eyeblink response to these singular or consecutive stimuli will be measured and scored to determine equivalent RSS from levels 3 through 6. The automated measurement of the RSS state will be accomplished concurrently with computation of the patient state index. The value of the computed RSS score, when displayed with the Patient State Index or BIS index, will enable an estimation of whether the index indicates natural sleep or drug induced hypnotic state. A change, especially an increase in the patient state index a few seconds after a provocative stimulus, provides an indication of patient responsiveness that can also be used to differentiate natural sleep from drug-induced hypnosis.

The numeric processed value of the response to the glabellar stimulator of the present invention can be displayed as a stand-alone trend or as a complementary independent indication of patient responsiveness to other processed hypnotic terms (such as PSI, BIS, State or Response Entropy) or unprocessed physiological parameters such as ETCO2, Blood Pressure or Heart Rate. The Ramsay Sedation Score, or any equivalent processed value, can be displayed as a dimensionless metric such as used in RSS or RASS, or probability score representative of the probability that the person is responsive or non-responsive.

Although the system of the present invention has been disclosed and described herein in the context of certain preferred embodiments, it will be appreciated by those of ordinary skill in the art that various modifications to and equivalents of the system can be made without departing from the intended spirit and scope of the present invention. The following claims are intended to encompass such modifications and equivalents.

What is claimed is:

1. A system for determining a patient's level of sedation, said system comprising
a) a glabellar stimulator constructed to generate an electrical stimulus;
b) an electrode electrically connected to said glabellar stimulator, said electrode constructed to deliver said electrical stimulus from said glabellar stimulator to a patient; and
c) a patient module constructed to detect an eyeblink response of a patient following delivery of said electrical stimulus to the patient, said patient module constructed to generate a signal indicative of at least one parameter of said eyeblink response, wherein said at least one parameter of said eyeblink response is indicative of a patient's level of sedation.

2. A system for determining a patient's level of sedation in accordance with claim 1, wherein said electrical stimulus generated by said glabellar stimulator is charge neutral.

3. A system for determining a patient's level of sedation in accordance with claim 1, wherein said electrical stimulus generated by said glabellar stimulator is biphasic.

4. A system for determining a patient's level of sedation in accordance with claim 1, wherein said electrical stimulus generated by said glabellar stimulator is triphasic.

5. A system for determining a patient's level of sedation in accordance with claim 1, wherein said electrical stimulus generated by said glabellar stimulator is mono-phasic.

6. A system for determining a patient's level of sedation in accordance with claim 1, wherein said electrode is constructed for placement in electrical contact with a patient's nasion.

7. A system for determining a patient's level of sedation, comprising:
a) a stimulus generating circuit constructed to generate a charge neutral stimulus;
b) an electrode array comprising a plurality of electrodes, at least one of said plurality of electrodes electrically connected to said stimulus generating circuit and constructed to deliver said charge neutral stimulus to a patient;
c) a system constructed to receive and analyze one or more neurophysiological signals from a patient following delivery of said charge neutral stimulus to the patient, said system generating a parameter characteristic of a patient's level of sedation based upon said one or more neurophysiological signals.

8. A system for determining a patient's level of sedation in accordance with claim 7, wherein said stimulus generating circuit is constructed to generate a pulse train that alternates electrical flow direction and magnitude.

9. A system for determining a patient's level of sedation in accordance with claim 7, wherein said system further comprises a subsystem for temporarily deactivating said system constructed to receive and analyze one or more neurophysiological signals during delivery of said charge neutral stimulus to a patient.

10. A system for differentiating between natural sleep and sedation in a patient in a drug induced hypnotic state, comprising:
a) a circuit constructed to generate a substantially charge neutral electrical stimulus;
b) at least one electrode electrically connected to said circuit, said at least one electrode constructed to deliver said substantially charge neutral electrical stimulus from said circuit to a patient;
c) at least one electrode constructed to detect one or more neurophysiological signals from a patient that are generated in response to said substantially charge neutral electrical stimulus; and
d) a module constructed to receive from said at least one electrode and analyze said one or more neurophysiological signals, said module constructed to produce one or more parameters representative of a patient's state of sedation.
